(19) United States
(12) Patent Application Publication
(10) Pub. No.: US 2010/0152409 A1
(43) Pub. Date: Jun. 17, 2010

(54) Triptycene Derivatives, Method for Synthesizing the Same and Application Thereof

(75) Inventors: Chin-Wen Chen, Danshuei Township (TW); I-Pin Fu, Zhonghe City (TW); Yu-Chi Tseng, Chungli City (TW); Jye-Shane Yang, Yangmei Town (TW); Jyu-Lun Yan, Hualien City (TW)

Correspondence Address: THOMAS, KAYDEN, HORSTEMEYER & RISLEY, LLP, 600 GALLERIA PARKWAY, S.E., STE 1500, ATLANTA, GA, 30339-5994, US

(73) Assignee: TAIWAN TEXTILE RESEARCH INSTITUTE, Tu-Chen City (TW)

(21) Appl. No.: 12/344,971

(22) Filed: Dec. 29, 2008

(30) Foreign Application Priority Data
Dec. 16, 2008 (TW) 97149002

(51) Int. Cl. C08G 69/26; C07C 69/734; C07C 59/72; C08G 63/66

(52) U.S. Cl. 528/285; 560/56; 562/466; 528/305; 528/288; 528/324; 528/318

(57) ABSTRACT

Triptycene derivatives and method for preparing the same are provided. The triptycene derivatives may be triptycene di-ester of the formula (I) or triptycene di-acid of the formula (II):

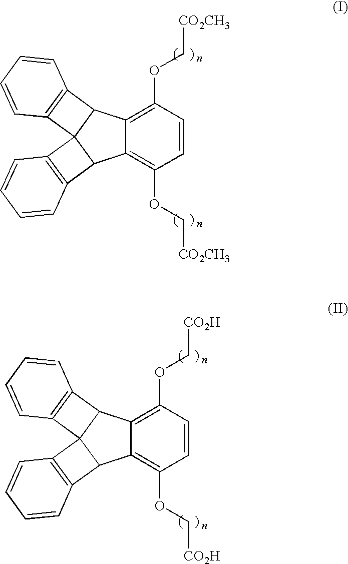

where n=1-5.

### RELATED APPLICATIONS

This application claims priority to Taiwan Application Serial Number 97149002, filed Dec. 16, 2008, which is herein incorporated by reference.

### BACKGROUND

1. Field of Invention

The present invention relates to triptycene derivatives and methods for synthesizing the same.

2. Description of Related Art

Modifying the properties of polymeric materials to enhance the industrial application thereof has been the goal of material industry.

Triptycene derivatives especially triptycene di-ol has unique structural property and thus can be incorporated into polymers to improve the toughness thereof. However, the method for synthesizing triptycene di-ol is complicated. Besides, triptycene di-ol can only be incorporated into polyester system and thus the application of triptycene di-ol in other polymer systems is limited.

In view of the foregoing, there is a need to provide a simple and easy method for synthesizing a triptycene derivative that is suitable to be incorporated into polymer systems in addition to polyester.

### SUMMARY

In one aspect, the present invention is directed to triptycene derivatives and methods for synthesizing the same. Said triptycene derivatives can be triptycene di-ester or triptycene di-acid. Said triptycene derivatives can be used as a modifying agent in copolymerization process to improve the physical property of the polymer.

According to one embodiment of the present invention, the method for synthesizing triptycene di-ester includes the procedure of heating a reaction system comprising triptycene di-ol, methyl bromocarboxylate, potassium iodide, potassium carbonate, and acetone. The resultant triptycene di-ester has a structure of the formula (I):

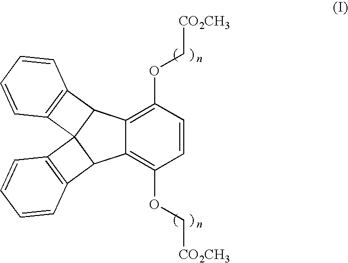

where n=1-5.

According to another embodiment of the present invention, the method for synthesizing triptycene di-acid includes adding the triptycene di-ester and about 3.5 wt % potassium hydroxide into tetrahydrofuran and stirring at room temperature, wherein the volume ratio of the tetrahydrofuran to the potassium hydroxide is about 1:1. The resultant triptycene di-acid has a structure of the formula (II):

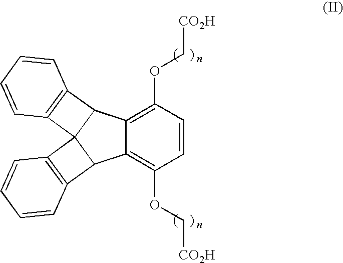

where n=1-5.

In another aspect, the present invention is directed to ductile polymers and a method for synthesizing the same. Said ductile polymer is copolymer of at least one polymerizable monomer and a triptycene derivative, the mass of the triptycene derivative is about 1-20% based on the total mass of the ductile copolymer, and wherein the triptycene derivative is triptycene di-ester of the formula (I) or triptycene di-acid of the formula (II):

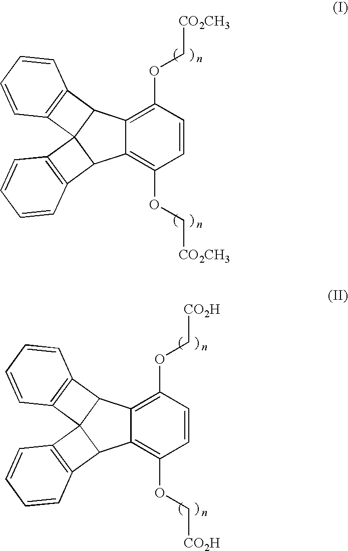

where n=1-5.

According to one embodiment of the present invention, the method for synthesizing the ductile polyester comprises copolymerizing at least one polymerizable monomer and a triptycene derivative under a copolymerization condition, wherein the mass of the triptycene derivative is about 1-20% based on the total mass of the ductile copolymer, and wherein the triptycene derivative is triptycene di-ester of the formula (I) or triptycene di-acid of the formula (II).

These and other features, aspects, and advantages of the present invention will become better understood with reference to the following description and appended claims.

### BRIEF DESCRIPTION OF THE DRAWINGS

The invention can be more fully understood by reading the following detailed description of the embodiment, with reference made to the accompanying drawings as follows:

FIG. 1 is a diagram showing the results of rheological analysis of ductile polyester according to one embodiment of the present invention.

### DETAILED DESCRIPTION

Reference will now be made in detail to the embodiments and examples of the present invention.

In one aspect, the present invention is directed to triptycene derivatives and methods for synthesizing the same. Method for synthesizing triptycene derivatives according to the embodiments of the present invention is described in detail in the following paragraphs. As will occur to those skilled in the art, the amount of the reactants, reaction time, and reaction temperature illustrated in the following embodiments can be adjusted without departing from the principals and spirits of the present invention as long as they fall within the limitation recited in the claim.

### (I) Triptycene Di-Ester and Method for Synthesizing the Same

According to one embodiment of the present invention, said triptycene derivative is triptycene di-ester. The method for synthesizing the triptycene di-ester according to this embodiment is simpler and easier than the known method. The resultant triptycene di-ester can be used to synthesize ductile polyester according to another aspect of the present invention.

According to one embodiment of the present invention, the method for synthesizing triptycene di-ester comprises heating a reaction system comprising: triptycene di-ol, methyl bromocarboxylate, potassium iodide, potassium carbonate, and acetone. The resultant triptycene di-ester has a structure of formula (I):

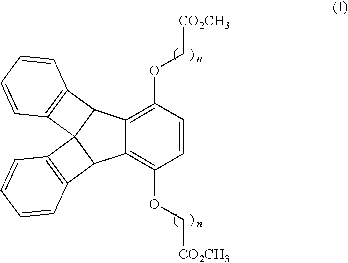

where n=1-5.

As an example but not limitation, the method for synthesizing triptycene di-ol includes the following procedures:

(1) to about 200 mL of toluene, about 28.0 g of anthracene (about 157 mmol) and about 34.0 g of benzoquinone (about 315 mmol) were added, the reaction mixture was heat-refluxed under nitrogen for about 4 hours and then cooled to room temperature;

(2) the reaction mixture of step (1) was filtered, and the thus obtained solid portion was washed with each 10 mL of toluene twice and then vacuum-dried to obtain a solid intermediate;

(3) about 40 g of the solid intermediate was dissolved in about 375 mL of hot acetic acid, then, about 4 mL of 48% hydrobromic acid aqueous solution was dropped in slowly, and the reaction mixture was allowed to stand overnight; and

(4) the reaction system of step (3) was filtered, and the thus obtained solid portion was washed sequentially with about 25 mL of acetic acid and about 50 mL of n-hexane and then dried to obtain the solid triptycene di-ol.

More specifically, the method for synthesizing triptycene di-ester according to the embodiments of the invention includes the steps of:

(1) to about 650 mL of acetone, about 18.0 g of triptycene di-ol (about 62.9 mmole), about 41.99 g of methyl bromoacetate (about 251.0 mmole, the molar ratio of triptycene di-ol: methyl bromoacetate is about 1:4), about 10.44 g of potassium iodide (about 62.9 mmole, the molar ratio of triptycene di-ol: potassium iodide is about 1:1), and 44.0 g of potassium carbonate (about 314.0 mmole, the molar ratio of triptycene di-ol: potassium carbonate is about 1:5), the reaction mixture was heat-refluxed to about 55-60° C. for about 6-24 hours and then cooled to room temperature;

(2) the reaction mixture of step (1) was filtered, and the thus obtained solid portion was washed with each 50 mL of acetone twice and then the filtrate was condensed to remove the solvent and to obtain a residue;

(3) to the residue of step (2), water and about 250 mL of dichloromethane was added, the solution was extracted and the organic layer was collected;

(4) the organic layer of step (3) was washed with saturated sodium chloride solution, and then the organic layer was dried by adding anhydrous magnesium sulfate thereinto; and

(5) the organic layer of step (5) was vacuum concentrated to obtain triptycene di-ester.

As will occur to those skilled in the art, methyl bromocarboxylate with different carbon numbers can be used to synthesize different triptycene di-ester. The methyl bromocarboxylate can be methyl bromoacetate, methyl bromopropionate, methyl bromobutyrate, methyl bromovalerate, or methyl bromocaproate.

### (II) Triptycene Di-Acid and Method for Synthesizing the Same

According to one embodiment of the present invention, said triptycene derivative is triptycene di-acid. The triptycene di-acid thus obtained is a novel compound, and can be used to synthesize ductile polyester and ductile polyamide according to another aspect of the present invention.

In one embodiment of the present invention, the method for synthesizing triptycene di-acid includes the following procedure. First, triptycene di-ester and about 3.5 wt % potassium hydroxide aqueous solution were added into tetrahydrofuran, wherein the volume ratio of tetrahydrofuran: potassium hydroxide aqueous solution is about 1:1. Then, the reaction mixture was stirred at room temperature to obtain triptycene di-acid.

The resultant triptycene di-acid has a structure of formula (II):

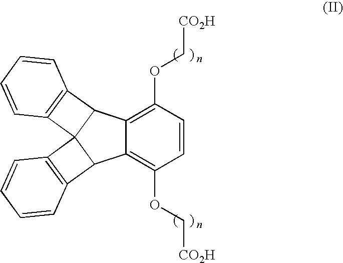

where n=1-5.

More specifically, the method for synthesizing triptycene di-acid according to the embodiments of the invention includes the steps of:

(1) to about 200 mL of tetrahydrofuran, about 19.5 g of triptycene di-ester (about 42.53 mmole) and about 3.5 wt % potassium hydroxide aqueous solution (about 7.4 g of potassium hydroxide dissolved in 200 mL of water), the reaction mixture was stirred at room temperature for about 1-6 hours;

(2) the reaction mixture of step (1) was vacuum concentrated to remove the solvent therein and the aqueous layer was collected;

(3) to the aqueous layer of step (2), concentrated hydrochloric acid was added until no solid precipitated anymore; and

(4) the precipitated solid of step (3) was filtered and the thus obtained solid was washed sequentially with about 100 mL of methanol and about 50 mL of n-hexane and then vacuum dried to obtain triptycene di-acid.

The resultant triptycene di-acid has a structure of formula (II):

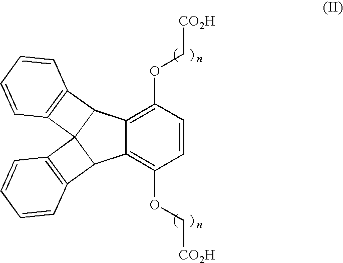

wherein, n=1, and is denominated in accordance with IUPAC nomenclature as 1,4-Bis(1-carboxymethoxy)-9,10-dihydro-9, 10[1′,2′]-benzenoanthracene.

As will occur to those skilled in the art, triptycene di-ester with different carbon numbers can be used to synthesize different triptycene ai-acid.

### (III) Ductile Polyester and Method for Synthesizing the Same

In another aspect, the present invention is directed to ductile polymers and a method for synthesizing the same. Said ductile polymer is a copolymer of at least one polymerizable monomer and a triptycene di-ester or triptycene di-acid mentioned above.

According to embodiments of the present invention, the method for synthesizing the ductile polyester comprises co-polymerizing at least one polymerizable monomer and a triptycene derivative under a copolymerization condition, wherein the mass of the triptycene derivative is about 1-20% based on the total mass of the ductile copolymer yields, and wherein the triptycene derivative is triptycene di-ester of the formula (I) or triptycene di-acid of the formula (II):

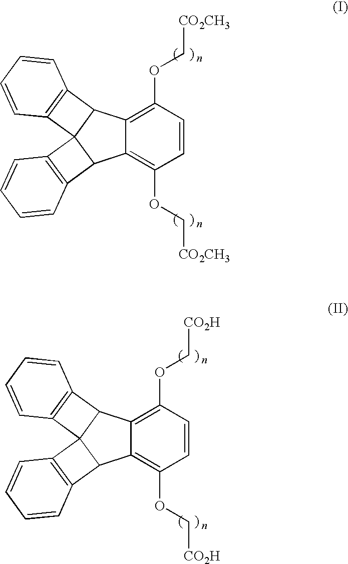

where n=1-5.

In one embodiment of the present invention, said ductile polymer can be ductile polyester. The ductile polyester is copolymer of monomers of polyester and triptycene derivative, wherein the mass of the triptycene derivative is about 1-20% based on the total mass of the ductile polyester, and wherein the triptycene derivative is triptycene di-ester of the formula (I) or triptycene di-acid of the formula (II).

Generally, monomers of polyester include alcoholic components and acid components. Just to name a few, alcoholic component can be ethylene glycol (glycol), propylene glycol, or 1,3-butanedi-ol . . . ; and acid component can be phthalic acid, isophthalic acid, terephthalic acid, or alkyl dicaboxylic acids . . . . As will occur to those skilled in the art, these and other suitable monomers of polyester can be used to synthesize the ductile polyester of the present invention without departing from the principals and spirits of the present invention.

More specifically, the monomers of polyester the method for synthesizing the ductile polyester includes the steps of:

(a) preparing a reaction system comprising terephthalic acid, ethylene glycol, and triptycene di-ester or triptycene di-acid, wherein the mass of triptycene di-ester or triptycene di-acid is about 1-20% based on the total mass of the ductile polyester; and

(b) copolymerizing the reaction system of step (a) under a copolymerization condition comprising: about 0.3-0.5 g of antimony acetate as a catalyst, a esterification temperature of at least about 260° C., a esterification pressure of about 2.5-3.5 bar, a polymerization temperature of at least about 280° C., and a vacuum level below about 5 mmHg.

In one specific embodiment of the present invention, the molar ratio of ethylene glycol to terephthalic acid is about 1.3-1.8. In this case, the reaction percentage of triptycene derivatives and the molecular weight of the ductile polyester are both desirable.

In the preparation examples listed in table 1, the amounts of the reactants were altered to synthesize ductile polyesters with various reactant ratios. More specifically, in examples 1 to 6, the triptycene derivative used was triptycene di-ester, esterification temperature was about 270° C., esterification pressure was about 2.7 bar, polymerization temperature was about 280° C.

TABLE 1

| | Terephthalic | Ethylene | Triptycene | | |
|---|---|---|---|---|---|
| Example | acid (g) | glycol (g) | di-ester (g) | | Yield (g) |
| 1 | 400 | 240 | 4.7 | (1 PHR) | 467.8 |
| 2 | 800 | 480 | 29 | (3 PHR) | 975.1 |
| 3 | 800 | 480 | 49 | (5 PHR) | 978.9 |
| 4 | 800 | 480 | 81 | (8 PHR) | 1013.9 |
| 5 | 800 | 480 | 104 | (10 PHR) | 1039.1 |
| 6 | 800 | 480 | 237 | (20 PHR) | 1184.7 |

In this specification, “PHR” denotes “parts per hundred ratio”, i.e., parts per 100 parts of ductile polymer obtained, on a weight basis. For example, the ductile polyester of example 6 has 20 PHR of triptycene di-ester means the mass of the triptycene di-ester to the total mass of the ductile polyester is about 20%.

From examples 1 to 6, it is appreciated that ductile polyester can be synthesized according to the methods provided in the embodiments of the present invention.

### (IV) Ductile Polyamide and Method for Synthesizing the Same

In another embodiment of the present invention, said ductile polymer can be ductile polyamide. The ductile polyamide is copolymer of monomers of polyamide and triptycene di-acid of the formula (II), wherein the mass of the triptycene di-acid is about 1-20% based on the total mass of the ductile polyamide:

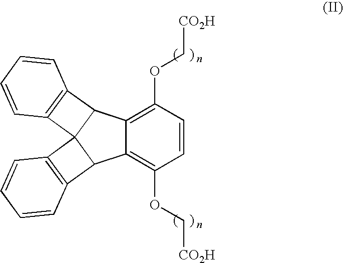

where n=1-5.

Generally, monomers of polyamide include, a caprolactam having a ring of not less than 3 carbons or a polymerizable omega-amino carboxylic acid, and a primary amine or other alkyl amine. As will occur to those skilled in the art, these and other suitable monomers of polyamide can be used to synthesize the ductile polyamide of the present invention without departing from the principals and spirits of the present invention.

More specifically, the method for synthesizing the ductile polyamide includes the steps of:

(a) preparing a reaction system comprising caprolactam, hexamethylenediamine, and triptycene di-acid, wherein the mass of triptycene di-acid is about 5-20% based on the total mass of the ductile polyamide; and

(b) copolymerizing the reaction system of step (a) under a copolymerization condition comprising: a ring-opening temperature of about 250° C., a ring-opening pressure of about 2 bar, a polymerization temperature of about 260° C., and a polymerization pressure of normal pressure.

According to embodiments of the present invention, the ring-opening reaction can proceed for about 3-3.5 hours, while the polymerization reaction can proceed for about 3.5-4.5 hours.

In the preparation examples listed in table 2, the amounts of the reactants were altered to synthesize ductile polyamides with various reactant ratios. More specifically, in examples 7 to 10, the triptycene derivative used was triptycene di-acid.

TABLE 2

| | Caprolactam | Hexamethylene | Triptycene | | Yield |
|---|---|---|---|---|---|
| Example | (g) | diamine (g) | di-acid (g) | | (g) |
| 7 | 450 | 6.93 | 24 | (5 PHR) | 478.8 |
| 8 | 450 | 11.55 | 40 | (8 PHR) | 498 |
| 9 | 450 | 14.58 | 51 | (10 PHR) | 511 |
| 10 | 450 | 34.5 | 119 | (20 PR) | 592.8 |

From examples 7 to 10, it is appreciated that ductile polyamide can be synthesized according to the methods provided in the embodiments of the present invention.

### (V) Stress-Strain Behavior and Spinnability of Ductile Polymer

Stress-strain behavior analysis was conducted to evaluate the toughness of the ductile polyesters of the examples of the present invention. The ductile polyesters of examples 1, 2, and 3 (PET-1, PET-2, and PET-3, respectively) were made into polymer chip for the analysis. Regular polyester (pure PET) and high intrinsic viscosity polyester (HV PET) were purchased from Shinkong textile (Taiwan) and were used as controls in the following analysis. The stress-strain behavior analysis was conducted in accordance with the CNS 4396 K6423 standard for test method of tensile stress of plastics. The plastics were made by injection-molding mechanic. Differential scanning calorimetry (DSC) was used to measure the melting point (Tm) of the polyesters samples. The results are listed in table 3.

TABLE 3

| | Pure PET | HV PET | PET-1 | PET-2 | PET-3 |
|---|---|---|---|---|---|
| Yield point | 32.55 | 35.87 | 35.68 | 38.85 | 41.13 |
| (Kgf/mm²) | | | | | |
| Break point | 52 | 57.32 | 58.44 | 59.98 | 64.93 |
| (Kgf/mm²) | | | | | |
| Strain (%) | 454.72 | 580.62 | 520.28 | 559.82 | 632.95 |
| Intrinsic viscosity | 0.64 | 1.0 | 0.77 | 0.73 | 0.69 |
| (IV) | | | | | |
| Tm (° C.) | 260 | 270 | 253.1 | 252.9 | 249.1 |

As can be seen in table 3, as compared with pure PET, the yield point of PET-3 of the present invention is increased by about 26.36%, the break point of PET-3 is increased by about 24.87, and the strain of PET-3 is increased by 39.20%.

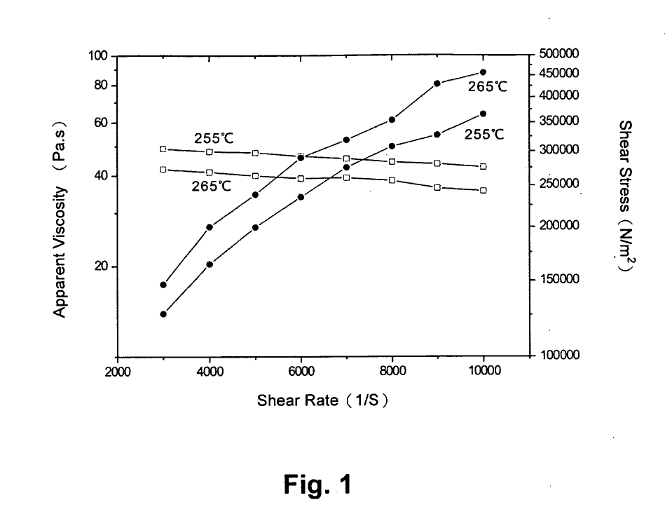

Further, the spinnability of the ductile of the embodiments of the present invention was evaluated by capillary rheometer (RG25, GÖffert. DE). Still refer to table 3, the ductile polyesters (PET-1, PET-2, and PET-3) have a Tm of about 249-254° C. Generally, the spinning temperature should be set about 20-30° C. higher than the Tm of the polymer to be spun. Therefore, the spinning temperature was set at 255° C. and 265° C. during the rheological analysis. FIG. 1 is a diagram showing the result of Theological analysis of PET-3.

Referring to FIG. 1, when the shear rate is about 3000-7000 1/s, the apparent viscosity of the ductile polyester PET-3 does not exhibit shear-thinning, and thus the ductile polyester PET-3 has suitable spinnability, and the spinning temperature can be set at about 265° C. In addition, temperature for melt-blown can be set at about 260° C.

In another aspect of the present invention, it is directed to a fabric made from material comprising the ductile polymer according to the embodiments of the present invention. Generally, textile is referred to fiber, yarn, cloth, and final product made therefrom. As it will occur to those skilled in the art, said ductile polymer can be made into polymer chip or polymer masterbatch, and the polymer chip or polymer masterbatch can be used by injection-molding, melt-blown, or spun into fabric.

It will be apparent to those skilled in the art that various modifications and variations can be made to the present invention without departing from the scope or spirit of the invention. In view of the foregoing, it is intended that the present invention cover modifications and variations of this invention provided they fall within the scope of the following claims.

What is claimed is:

1. A triptycene derivative of the formula (I) or of the formula (I): 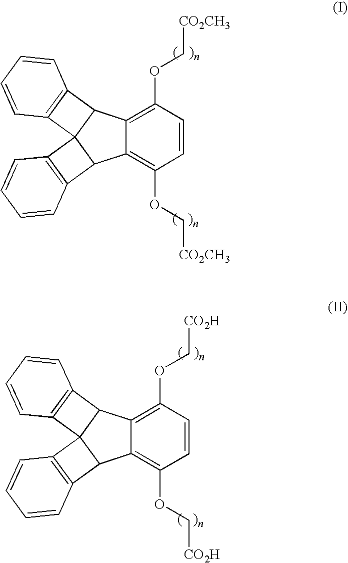
where n=1-5.

2. The triptycene derivative of claim 1, wherein n=1.

3. A method for synthesizing a triptycene derivative, comprising (a) heating a reaction system to obtain triptycene di-ester, wherein the reaction system comprises: triptycene di-ol, methyl bromocarboxylate, potassium iodide, potassium carbonate, and acetone.

4. The method for synthesizing a triptycene derivative of claim 3, further comprising (b) adding the triptycene di-ester and 3.5 wt % potassium hydroxide into tetrahydrofuran and stirring at room temperature, wherein the volume ratio of the tetrahydrofuran to the potassium hydroxide is about 1:1.

5. The method for synthesizing a triptycene derivative of claim 3, wherein the molar ratio of triptycene di-ol: methyl bromocarboxylate: potassium iodide: potassium carbonate is about 1:4:1:5.

6. The method for synthesizing a triptycene derivative of claim 3, wherein the methyl bromocarboxylate is methyl bromoacetate, methyl bromopropionate, methyl bromobutyrate, methyl bromovalerate, or methyl bromocaproate.

7. The method for synthesizing a triptycene derivative of claim 3, wherein in the step (a), the reaction system is heat-refluxed to about 55-60° C.

8. The method for synthesizing a triptycene derivative of claim 3 wherein in the step (a), the reaction system is heated for about 6-24 hours.

9. The method for synthesizing a triptycene derivative of claim 3, wherein after the step (a), the reaction system is cooled to room temperature.

10. The method for synthesizing a triptycene derivative of claim 3, wherein the step (b) is performed for about 1-6 hours.

11. A ductile copolymer of at least one polymerizable monomer and a triptycene derivative, wherein the mass of the triptycene derivative is about 1-20% based on the total mass of the ductile copolymer, and wherein the triptycene derivative is triptycene di-ester of the formula (I) or triptycene di-acid of the formula (II): 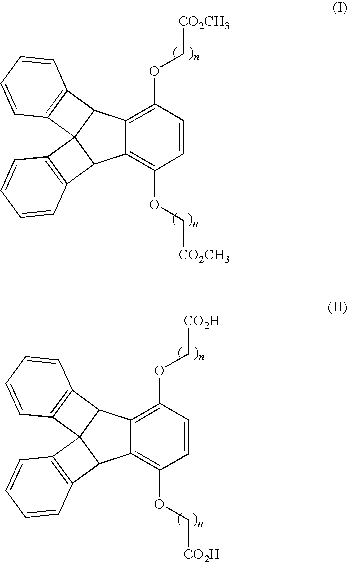
where n=1-5.

12. The ductile polymer of claim 11, wherein the polymerizable monomer is monomers of polyester and the triptycene derivative is triptycene di-ester of the formula (I) or triptycene di-acid of the formula (II).

13. The ductile polymer of claim 11, wherein the polymerizable monomer is monomers of polyamide, the triptycene derivative is triptycene di-acid of the formula (II), and the mass of the triptycene di-acid is about 5-20% based on the total mass of the ductile copolymer.

14. A method for synthesizing a ductile polymer, comprising co-polymerizing at least one polymerizable monomer and a triptycene derivative under a copolymerization condition, wherein the mass of the triptycene derivative is about 1-20% based on the total mass of the ductile copolymer, and wherein the triptycene derivative is triptycene di-ester of the formula (I) or triptycene di-acid of the formula (II): 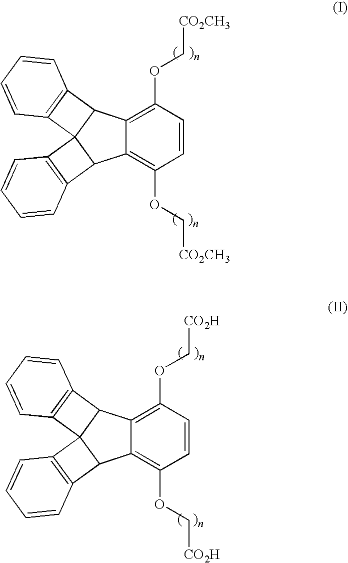
where n=1-5.

15. The method for synthesizing a ductile polymer of claim 14, wherein the polymerizable monomer comprises monomers of polyester, and the triptycene derivative is triptycene di-ester of the formula (I) or triptycene di-acid of the formula (II).

16. The method for synthesizing a ductile polymer of claim 15, wherein the monomers of polyester comprises terephthalic acid and ethylene glycol.

17. The method for synthesizing a ductile polymer of claim 16, wherein the molar ratio of terephthalic acid: ethylene glycol is about 1.3-1.6.

18. The method for synthesizing a ductile polymer of claim 15, wherein the copolymerization condition comprises: about 0.3-0.5 g of antimony acetate as a catalyst, a esterification temperature of at least about 260° C., a esterification pressure of about 2.5-3.5 bar, a polymerization temperature of at least about 280° C., and a vacuum level of at most about 5 mmHg.

19. The method for synthesizing a ductile polymer of claim 14, wherein the polymerizable monomer comprises monomers of polyamide, the triptycene derivative is triptycene di-acid of the formula (II), and the mass of the triptycene di-acid is about 5-20% based on the total mass of the ductile copolymer.

20. The method for synthesizing a ductile polymer of claim 19, wherein the monomers of polyamide comprises caprolactam and hexamethylenediamine.

21. The method for synthesizing a ductile polymer of claim 19, wherein the copolymerization condition comprises: a ring-opening temperature of about 250° C., a ring-opening pressure of about 2 bar, a polymerization temperature of about 260° C., and a polymerization pressure of normal pressure.
